(19) United States
(12) Patent Application Publication
(10) Pub. No.: US 2026/0009792 A1
(43) Pub. Date: Jan. 8, 2026

(54) CARRIER, CARRIER SYSTEM AND METHOD

(71) Applicant: SEMARION LTD, Cambridge (GB)

(72) Inventors: Jeroen Verheyen, Cambridge (GB); Tarun Vemulkar, Cambridge (GB)

(21) Appl. No.: 19/136,635

(22) PCT Filed: Dec. 7, 2023
(86) PCT No.: PCT/GB2023/053155

§ 371 Date: Sep. 16, 2025

(30) Foreign Application Priority Data
Dec. 8, 2022 (GB) 2218477.4
Dec. 8, 2022 (GB) 2218480.8

(51) Int. Cl. G01N 33/543; G01N 33/553

(52) U.S. Cl. CPC G01N 33/54393; G01N 33/553

(57) ABSTRACT

A carrier (100) for an adherent assay sample is provided. The carrier comprises a sample surface (25) for receiving the adherent sample and a peripheral region (28) between the sample surface and an edge of the carrier. The peripheral region comprises a sample-adhesion-reducing coating such that, in use, the adherent sample adheres less strongly to the peripheral region than to the sample surface.

The invention relates to a carrier, carrier system and method, and in particular to a carrier system for an assay, and to methods of manufacturing the carrier system.

### Background Art

An assay is an investigative procedure for qualitatively or quantitatively measuring the presence, amount or activity of an analyte in an assay sample. Often, the assay sample is a cell or a cell culture and the analyte is a protein or gene sequence of that cell or cell culture, but analyses may more generally include metabolites, peptides, proteins, nucleic acids, extracellular vesicles, organelles, cells, or tissues.

Some biological assay samples, such as certain types of cells, may be suspended in an aqueous solution. However, there are many biological assay samples which cannot easily be suspended in solution and which are most viable when attached to a surface, such as adherent cells. Assays of such samples, including the step of measuring, typically have to be performed while the assay sample is attached to the bottom of a well plate. In such assays, the assay process may be disadvantageously limited by the fact that an assay sample attached to the bottom of a well plate cannot easily be transferred to another container without stripping it from the well plate. This may damage the sample.

An improved approach to carrying out an assay using adherent assay samples, such as adherent cells, is to use a carrier or particle for carrying the assay sample. Examples of such carrier systems are described in patent publications WO2020/099846 and WO2021/224631.

In such a system a plurality of carriers can be provided secured to a substrate and adherent cells can be received onto the exposed surfaces of the carriers, for example from a biocompatible solution. The carriers may be magnetic; they can then be held (by an applied magnetic field) onto the substrate to allow time for the samples to adhere to the carriers. Subsequently, the magnetic carriers together with the adherent samples can be released from the substrate and directed or steered through an assay by an applied magnetic field.

However, when adherent samples are received onto carriers, particularly if multiple carriers are positioned close to each other on a substrate, a problem arises in that samples may adhere to or migrate to the substrate, or a sample may adhere to both the substrate and a carrier.

Approaches to spatially constraining cell growth are known in certain applications.

Cells may conventionally be grown or cultured on poly (dimethyl siloxane) (PDMS) substrates, as in “Large area micropatterning of cells on polydimethylsiloxane surfaces”, Moustafa et al, Journal of Biological Engineering 2014, 8:24. This method does not involve the use of carriers, but describes a method for spatial control and patterning of cell growth on a PDMS substrate. In this method, patterned barriers to cell adhesion are formed on the substrate using photo-activated graft polymerisation of polyethylene glycol, diacrylate (PEG-DA). This encourages cell growth within defined microchannels between the barriers.

Another approach is described in “Micropallet arrays with poly (ethylene glycol) walls”, Wang et al, in The Royal Society of Chemistry's Lab Chip, 2008, 8, 734-740. Wang et al fabricated an array of microwells on a glass substrate, each microwell containing a planar micropallet of SU-8 at its base. Adherent cells could be received into the microwells, onto the micropallets, and then individual micropallets could be driven out of their microwells by a focussed laser pulse directed through the glass substrate beneath any required micropallet. The cells carried by the released micropallet could then be collected, cultured and clonally expanded. Wang et al describe two approaches to forming the microwells. One was to form virtual air walls between the micropallets, by applying a hydrophobic coating to the glass substrate and forming a bubble which adheres to the substrate by Cassie-Baxter wetting. The other was to form photopolymerised PEG hydrogel walls on the glass substrate, in a similar way to Moustafa et al but defining the PEG walls between the micropallets by using the difference between the UV transmittance of the glass substrate and the SU-8 micropallets to localise the photopolymerisation. Individual micropallets could still be detached from between the walls on the substrate by using a focussed laser pulse.

However, when magnetically-steerable carriers such as those in WO2020/099846 and WO2021/224631 are used, several problems arise if these approaches to preventing cell attachment to the substrate are used. One is that the presence of walls on the substrate between the carriers may block the release of the carriers from the substrate. Wang et al used laser pulses to positively drive their micropallets from the substrate. But if the carriers, or micropallets, are simply released from the substrate by removing an applied magnetic field they may be retained in place by the walls. Another problem is that Wang et al's approach requires the use of a transparent glass substrate both to provide a photomask to enable wall formation and to enable micropallet detachment using laser pulses.

A further problem in the conventional use of carriers for adherent assay samples arises when magnetic carriers are used, and the carriers are directed or steered through an assay by an applied magnetic field. Under these circumstances, the adherent assay samples can be subjected to significant shear forces as they are driven through the assay medium (typically a biocompatible solution). This is not problematic for many assay samples, but for larger assay samples with lower levels of adhesion, the shear forces can be detrimental to the assay results.

An object of the invention is to solve these problems.

### SUMMARY OF INVENTION

The invention advantageously provides a carrier for an adherent assay sample, a carrier system and a method as defined in the appended independent claims, to which reference should now be made. Preferred or advantageous features of the invention are set out in dependent subclaims.

In a first aspect, the invention may thus provide a carrier for an adherent assay sample. The carrier may comprise a sample surface for receiving the adherent sample and a peripheral region surrounding the sample surface and positioned between the sample surface and an edge of the carrier. At least a portion of the carrier comprises a coating or coatings such that the adherent sample adheres less strongly to the peripheral region than to the sample surface. Suitable coatings to achieve this may be applied to the sample surface, or to the peripheral region, or to both the sample surface and the peripheral region. Preferably, the peripheral region comprises a sample-adhesion-reducing coating.

The presence of the coating(s) on the carrier may advantageously encourage the adherent sample to adhere to the sample surface, which forms a defined area away from, or spaced from, the edges of the carrier.

The coating(s) may advantageously ensure that the adherent sample adheres, in use, more strongly to the sample surface than to the peripheral region. This may advantageously mean that a sample is adhered only to a defined region of the carrier surface, which may make analysis of the sample-adhered carriers easier. For example it may be simpler to analyse a sample adhered to a central sample surface of a carrier which is easily viewable from above, rather than a sample adhered to the perimeter or edges of a carrier.

A surface of a carrier comprising the sample surface and the peripheral region is preferably flat, or planar, or substantially flat or planar. For example, a body or base layer of the carrier may comprise a flat or planar surface for carrying the sample, which may then receive surface treatments, or be coated, to increase or encourage sample adhesion to the sample surface and/or to reduce or discourage sample adhesion to the peripheral region.

Surface treatments such as coatings to the sample surface and/or to the peripheral region may be of different thicknesses, but within the scope of embodiments of the invention the resulting surfaces may be considered substantially flat or planar. In other words, any difference in height between the sample surface and the peripheral region is preferably less than 50% of the maximum thickness of the coated carrier, and particularly preferably less than 20% or 10% of the maximum thickness of the coated carrier.

The peripheral region may be raised relative to the sample surface. Where the peripheral region is raised relative to the sample surface, the raised peripheral region may additionally present a physical obstacle to cells if they tend to move away from the sample surface. This physical obstacle may enhance the effect of the sample-adhesion-reducing coating in retaining the cells on the sample surface. For example, the peripheral region may then be termed, or comprise, a wall for restraining or encircling cells adhering to the sample surface. The carrier may comprise a wall between the sample surface and an edge of the carrier, for encouraging the adherent sample to adhere to the sample surface and impeding movement of the adherent sample away from the sample surface. To achieve this, the geometrical shape of the wall may be sufficient to impede movement or adhesion of the sample on surfaces other than the sample surface, but it may also be advantageous if the adherent sample adheres, in use, less strongly to the wall than to the sample surface.

The carrier may advantageously be positioned or positionable on a substrate for receiving the adherent assay sample. In that case, when the carrier is positioned on the substrate the peripheral region, for example the wall, is preferably between the sample surface and the substrate.

The peripheral region preferably completely surrounds the sample surface, in a continuous loop or ring around the perimeter of the carrier, and preferably extends from the carrier edge to an outer perimeter of the sample surface. Particularly preferably, in embodiments comprising a wall, the wall surrounds or encircles the sample surface

Advantageously, a coating is applied to the peripheral region of the carrier, and the coating is configured to reduce or prevent adhesion of sample to the peripheral region, such that the adherent sample adheres less strongly to the peripheral region than to the sample surface. The coating for the peripheral region may thus be termed a sample-repellent coating, a non-adherent coating or an adhesion-reducing coating. As the sample is typically biological cells of some type, the coating is preferably a coating that reduces cell-adhesion to the peripheral region of the carrier.

A coating applied to the peripheral region may be a non-ionic surfactant. In preferred embodiments, the peripheral region may be coated with one or more of Pluronic F-127, or Poly-HEMA, or other Poloxamers.

The base or body of the carrier, to which the coating is applied, may be fabricated in any convenient manner. In a preferred embodiment the base is lithographically fabricated. In such a case, the base including its peripheral borders beneath the peripheral region may comprise a photoresist such as SU-8. A central portion of the base of the carrier may then be selectively treated, or coated, for example with metal layers (such as magnetic or gold layers) using a second lithography step.

Alternatively, the base or body of the carrier may be formed using different materials for the central portion beneath the sample surface, and for the peripheral portion beneath the peripheral region.

Where the peripheral region of the carrier comprises a photoresist material such as SU-8,it may be oxygen plasma treated and then coated with silane terminated PEG molecules, so that the silanes covalently bind to the O₂-plasma treated surface and the PEG molecules form an anti-stick coating.

Providing a non-sample-adherent peripheral region may advantageously decrease the likelihood of sample cells migrating between nearby carriers in use, as the peripheral region may create a spatial separation between the sample surfaces of adjacent carriers when the carriers are positioned on a substrate. In the event that carriers are ever positioned on the substrate in an overlapping manner, with the edge of one carrier overlapping the edge of another (which may occur for example if multiple carriers are deposited or magnetically steered onto a substrate during an assay), the peripheral region also improves the chances that the sample surfaces of each carrier will remain visible from above during analysis.

In embodiments of the invention comprising a wall, the geometrical shape of the wall may help to constrain an adherent sample to the sample surface, but the geometrical effect by itself is unlikely to be sufficient. At least a portion of a surface of the wall may therefore advantageously comprise a surface treatment, such as a coating, for example the sample-adhesion-reducing coating, such that the adherent sample adheres less strongly to the wall than to the sample surface. For example, the wall or a portion of the wall may be coated with a non-ionic surfactant. The wall or a portion of the wall may be coated with Pluronic F-127, Poly-HEMA, or other Poloxamers. Where the wall comprises a photoresist material such as SU-8, the wall may be oxygen plasma treated and then coated with silane terminated PEG molecules, so that the silanes covalently bind to the O₂-plasma treated surface and the PEG molecules form an anti-stick coating.

Alternatively to, or in addition to, providing a sample-adhesion-reducing coating on the peripheral region, the sample surface may comprise a coating which is configured so that that the adherent sample adheres more strongly to the sample surface than to the peripheral region. This may help to constrain an adherent sample to the sample surface. Such a sample-surface coating may be termed an adhesion-promoting coating. For example, the sample surface may comprise a bio-functionalised coating configured to attach to or adhere to the assay sample. The sample surface may comprise a charged coating. This may be particularly preferable when the assay sample is inherently charged. For example, cells typically have a membrane potential of between negative 40 and negative 80 millivolts. Providing a coating defining a surface having a positive charge may encourage attachment of the assay sample onto the charged surface of the carrier. It has been found that providing a charged surface, and preferably a positively charged surface, may advantageously lead to more effective cell adhesion. The coating may comprise a charged polymer.

In some embodiments, the sample surface may comprise a gold layer in the form of a gold cap, and the adhesion-promoting coating may be applied on the gold layer. The gold cap may be formed only in the region of the carrier which forms the sample surface. If the adhesion-promoting coating is then applied on the gold layer, only the sample surface of the carrier is bio-functionalized and so only the sample surface of the carrier tends to receive the assay sample, while the peripheral region (which does not comprise the functionalised gold cap) does not receive the assay sample.

The polymer (charged polymer) may be covalently bound to the gold cap forming the sample surface. The sample surface adapted for receiving the assay sample may comprise a gold cap layer on to which a polymer is covalently bonded by a thiol group. This advantageously ensures that each polymer adsorbed onto the surface has the same orientation. Alternatively, the polymer may be adsorbed onto the gold cap of the carrier by Van der Waals forces.

The charged polymer may be a polymer comprising a positive-charge-carrying group. The polymer may be a polyornithine or a poly-d-lysine. The polymer may be a polyelectrolyte.

Alternatively or additionally, the coating of the sample surface may comprise a plurality of ligands. The plurality of ligands may comprise antibodies. When the assay sample of the assay is a cell, the plurality of ligands may comprise antibodies that specifically bind to cell receptors such as integrins.

When the assay sample is a cell, the sample-surface coating may alternatively or additionally comprise an extracellular matrix protein. The extracellular matrix protein may be a protein selected to increase or enhance cell adhesion. The protein may be a collagen, laminin or Matrigel, a protein mixture secreted by Engelbreth-Holm-Swarm (EHS) mouse sarcoma cells.

In a preferred embodiment, the sample surface may be substantially flat, and is preferably parallel to the substrate when the carrier is positioned on a substrate. The or each carrier may advantageously comprise a body, or base portion, which is preferably laminar and may be of a high-aspect-ratio shape so that its lateral dimensions, or its length and breadth, may be many times greater than its thickness, for example 500 or 1000 times more than its thickness. An upper surface of the base portion (for example one of the larger surfaces of the high-aspect-ratio shape) may then form or carry the sample surface. The peripheral region is preferably also provided on the upper surface of the base portion, in substantially the same plane as (or coplanar with) the sample surface. Alternatively, the wall may extend upwardly from the upper surface of the base portion, adjacent to or spaced from the sample surface. As noted above, the sample surface and the peripheral region may carry surface treatments, such as coatings, of different thicknesses so that they may not be exactly in the same plane.

The carrier is preferably a planar particle. The carrier particle preferably comprises a planar first face, and a planar second face arranged parallel to the first face on the opposite side of the carrier particle. The sample surface and the peripheral region are preferably provided on the same face of the carrier, for example both on the first face, while the other face may be attached to a release layer for example. Due to the presence of the peripheral region, the sample surface may not occupy the entire face of the carrier, and the sample surface may not extend to the edge of the carrier.

To achieve the desired effect of confining a sample away from the carrier edges to discourage sample migration to other carriers, at least a portion of the peripheral region preferably has a width of greater than 1 micrometre and less than 50 micrometres, or greater than 5 micrometres and less than 30 micrometres, preferably greater than 8 micrometres and less than 25 micrometres. For example, the peripheral region may have a width of 10 micrometres. Preferably the peripheral region has a uniform width around the sample surface.

To achieve the desired geometrical effect of the wall, at least a portion of the wall may have a height above the sample surface of greater than 1 micrometre, or 2 micrometres, or 3 micrometres, and less than 50 micrometres, or 20 micrometres, or 15 micrometres. Also, at least a portion of the wall may have a lateral thickness, or a maximum lateral thickness, of greater than 1 micrometre and less than 50 micrometres, preferably greater than 5 micrometres and less than 40 micrometres.

To modify the effect of the wall to constrain or contain the adherent sample, at least a portion of the wall may have a rectangular cross section, or may have a flared or rounded cross section. For example, the cross-section of at least a portion of the wall may be stepped, or its thickness may vary non-monotonically. In particular, the cross-section of the wall may be flared such that a cross-section of the wall further from the sample surface is greater than the cross-section of the wall nearest to the sample surface. For example a portion of the wall may overhang the sample surface. This may improve the containment of the adherent sample. Alternatively, the cross-section of the wall may be flared such that the cross-section of the wall further from the sample surface is smaller than the cross-section of the wall nearest to the sample surface. This may allow the adherent cells to reach the sample surface of the base portion more easily. The wall preferably has the same cross section throughout its length.

Flared walls where the top is thicker than the base of the walls, or walls which overhang the sample surface, may advantageously provide a mechanical barrier to cell movement away from the sample surface. Alternatively, flared walls where the top of the wall is less thick than the base of the walls may advantageously allow any cells which may adhere to the wall to migrate down to the sample surface. The wall shape may thus be optimised for different cell or sample types, and different wall and sample-surface coatings.

Advantageously, the carrier may have a lateral dimension, preferably a minimum lateral dimension, of greater than 5 micrometres and less than 300 micrometres, or preferably greater than 10 micrometres and less than 200 micrometres.

The portion of the carrier beneath the peripheral region may be fabricated by any suitable method, but advantageously, for convenience and efficiency, it may comprise a material or materials also present in the portion of the carrier beneath the sample surface. The portion of the carrier beneath the peripheral region is preferably fabricated at the same time as, or in the same process as, the portion of the base beneath the sample surface. These portions of the base of the carrier may be of the same material(s) which extend continuously beneath both the peripheral region and the sample surface.

For example, the base portion may conveniently be fabricated using a lithographic process, in which case a magnetic material can also be incorporated into the carrier.

In a preferred embodiment, the peripheral region is simply a coated region of the base portion of the carrier.

The peripheral region of the carrier may be fabricated using nanoimprint or micro-imprint lithography.

In a preferred embodiment, the sample surface is simply a coated region of the base portion of the carrier.

In one embodiment, particularly if the carrier is made using photolithography, the portion of the carrier base beneath at least the peripheral region may comprise a structural photopolymer, preferably a photoresist, SU-8 or AZ-10nXT. Alternatively, or in addition, the portion of the base beneath at least the peripheral region may comprise a polymer, such as a thermoplastic polymer, preferably PDMS.

The wall of the carrier may be fabricated by any suitable method, but advantageously, for convenience and efficiency, the wall may comprise a material which is also present in the base portion, and the wall is preferably fabricated using a process which is also used to fabricate the base portion. For example, the wall and the base portion may conveniently be fabricated using a lithographic process, in which case a magnetic material can also be incorporated into the carrier.

The wall may be fabricated using nanoimprint lithography or micro-imprint lithography.

In one embodiment, particularly if the carrier is made using photolithography, the wall may comprise a structural photopolymer, preferably a photoresist, SU-8 or AZ-10nXT. Alternatively, or in addition, the wall may comprise a polymer, such as a thermoplastic polymer, preferably PDMS.

In a further aspect, the invention may advantageously provide a carrier system for an assay, comprising a plurality of carriers. In the carrier system, the carriers are preferably initially provided secured to a substrate by, for example, a release layer. The carriers on the substrate may then be exposed to a biocompatible solution in which adherent assay samples, such as adherent cells, are suspended, so that the adherent samples can adhere to the sample surfaces of the carriers. The release layer may be activated by, or may dissolve in, the biocompatible solution so that the carriers carrying the adherent samples are released from the substrate into the solution. Advantageously, during an assay the adherent samples may be carried on the sample surface of each carrier, and constrained from bridging or crossing from the sample surface to the substrate by the peripheral region, for example by the wall, separating the sample surface from the edge of the carrier.

In a further preferred embodiment, the substrate between the carriers may additionally receive a surface treatment, or be coated, to reduce adhesion to adherent samples and so further reduce any risk that samples may bridge or cross from carriers to the substrate.

For example, during fabrication of the carrier system the release layer is preferably applied to the whole substrate, including in areas between the (subsequently-formed) carriers. In one embodiment, the release layer between carriers may be removed after at least the carrier base portions have been formed, and the underlying substrate treated or coated to reduce cell adhesion.

It may not be appropriate to apply such a surface treatment or coating to the release layer itself, because the release layer typically dissolves in the biocompatible solution as the adherent samples are loaded onto the sample surfaces of the carriers.

The carrier may advantageously be magnetic, or comprise a magnetic material. In that case it can be held on the substrate by an applied magnetic field even after the release layer has been dissolved, to allow more time for the adherent samples to adhere to the sample surfaces.

In yet a further aspect, the invention may advantageously provide a method of performing an assay using the carrier or the carrier system as described above. The method may advantageously comprise the step of providing a plurality of carriers positioned on a substrate, introducing a biocompatible aqueous solution carrying the adherent samples to the carriers and the substrate so that the adherent samples adhere to the sample surfaces of the carriers but not to the peripheral regions of the carriers, and releasing the carriers from the substrate. The carriers may be held in position on the substrate by a release layer between the carriers and the substrate, the release layer being activated by the biocompatible aqueous solution to release the carriers. In addition, or in the alternative, the carriers may be magnetic or comprise a magnetic material and may then be held in position on the substrate by an applied magnetic field and released from the substrate by changing or removing the applied magnetic field.

In yet a further aspect of the invention, it should be noted that the carrier of the invention is preferably magnetically steerable, for example through the stages of an assay. Therefore, after release from the substrate, a solution in an assay may contain a large number of carriers, carrying adherent samples. While in solution, the carriers may move near to each other and it is important to avoid the adherent samples moving from one carrier to another. The sample-repellent effect of the peripheral region, surrounding the adherent samples on each carrier, may advantageously assist in keeping the sample surfaces of different carriers spaced from each other when the carriers are in solution, and thus may reduce the risk of adherent samples moving from one carrier to another.

In some cases, individual carriers may comprise a readable code, or otherwise be marked so that individual carriers, or groups of carriers, can be identified in an assay. If adherent samples are placed on an individual carrier at the start of an assay, then it is then essential that those samples do not move from one carrier to another. The coated peripheral regions surrounding the samples on carriers in solution, which may move near to or past other carriers, may advantageously prevent movement of samples between carriers.

The readable code, such as a barcode or QR code, may for example be applied to a surface of the carrier, or it may comprise a hole or a number of holes defined through or within the base portion of the carrier. Preferably, the code may be located in the peripheral region. Advantageously, this may prevent the code from reducing the area of the sample surface available for cell attachment. Preferably, the wall comprises the readable code. Advantageously, the readable code being comprised in the wall prevents the code from reducing the area of the sample surface available for cell attachment. The code may penetrate partially into the wall. The code may penetrate through the wall to the carrier base. The readable code may comprise a number of dots, dashes, or dots and dashes. The code may comprise a number of lines. The code may be formed by lithography.

The readable code may take a variety of forms as markings in the carrier, which encode information identifying the carrier to which that readable code is applied. When imaged, the pattern in a given carrier can be analysed for its coding information, and thus used to identify that specific carrier. The code is preferably readable by image analysis of a microscopy image.

Also, in an assay, magnetically-steerable carriers may commonly be steered onto a substrate, for example for imaging or analysis. When the carriers are placed onto a substrate in this way, the coated peripheral regions of the carriers may advantageously impede adherent samples from moving from the carriers to the substrate, or from one carrier to another.

As noted above, the sample surface and the peripheral region of a carrier embodying the invention may be coplanar, or they may optionally not be coplanar. One option is for the peripheral region to be raised relative to the sample surface. This aspect of the invention may advantageously relate to the further aspect of the invention discussed below.

In this further aspect, the invention may thus provide a carrier for an adherent assay sample. The carrier may comprise a sample surface for receiving the adherent sample and a wall between the sample surface and an edge of the carrier, for encouraging the adherent sample to adhere to the sample surface and impeding movement of the adherent sample away from the sample surface. To achieve this, the geometrical shape of the wall may be sufficient to impede movement or adhesion of the sample on surfaces other than the sample surface, but it may also be advantageous if the adherent sample adheres, in use, more strongly to the sample surface than to the wall.

The carrier may advantageously be positionable on a substrate for receiving the adherent assay sample. In that case, when the carrier is positioned on the substrate the wall is preferably between the sample surface and the substrate.

Particularly preferably, the wall surrounds or encircles the sample surface.

In a preferred embodiment the carrier may form part of a carrier system in which a plurality of carriers is provided on a substrate, and preferably secured to the substrate by, for example, a release layer. The carriers on the substrate may then be exposed to a biocompatible solution in which the adherent assay samples, such as adherent cells, are suspended, so that the adherent samples can adhere to the sample surfaces of the carriers. The release layer may be activated by, or may dissolve in, the biocompatible solution so that the carriers carrying the adherent samples are released from the substrate into the solution. Advantageously, the adherent samples may be carried on the sample surface of each carrier, and constrained from bridging or crossing from the sample surface to the substrate by the wall.

The carrier may advantageously be magnetic, or comprise a magnetic material. In that case it can be held on the substrate by an applied magnetic field even after the release layer has been dissolved, to allow more time for the adherent samples to adhere to the sample surfaces.

In a preferred embodiment, the sample surface is substantially flat, and is preferably parallel to the substrate when the carrier is positioned on the substrate. The or each carrier may advantageously comprise a base portion, which is preferably laminar and may be of a high-aspect-ratio shape so that its lateral dimensions (length and breadth) may be many times greater than its thickness, for example 500 or 1000 times more than its thickness. An upper surface of the base portion (for example one of the larger surfaces of the high-aspect-ratio shape) may then form or carry the sample surface, and the wall may extend upwardly from the upper surface of the base portion, adjacent to or spaced from the sample surface.

Although the geometrical shape of the wall may be sufficient to constrain an adherent sample to the sample surface, for some types of adherent sample the geometrical effect may not be sufficient. Advantageously, therefore, at least a portion of the sample surface may comprise a surface treatment, such as a coating, such that the adherent sample adheres more strongly to the sample surface than to the wall. For example, the sample surface may comprise a gold cap layer on to which a polymer is covalently bonded by a thiol group. The polymer may be a polymer comprising a positive-charge-carrying group. The polymer may be a polyornithine or a poly-d-lysine. The polymer may be a polyelectrolyte.

Alternatively, or in addition, at least a portion of a surface of the wall may comprise a surface treatment, such as a coating, such that the adherent sample adheres less strongly to the wall than to the sample surface. For example, the wall or a portion of the wall may be coated with a non-ionic surfactant. The wall or a portion of the wall may be coated with Pluronic F-127, Poly-HEMA, or other Poloxamers. Where the wall comprises a photoresist material such as SU-8, the wall may be oxygen plasma treated and then coated with silane terminated PEG molecules, so that the silanes covalently bind to the O2-plasma treated surface and the PEG molecules form an anti-stick coating.

To achieve the desired geometrical effect of the wall, at least a portion of the wall may have a height above the sample surface of greater than 1 micrometre and less than 50micrometres, preferably greater than 2 micrometres and less than 20 micrometres, or greater than 3 micrometres and less than 15 micrometres. Also, at least a portion of the wall may have a lateral thickness, or a maximum lateral thickness, of greater than 1micrometre and less than 50 micrometres, preferably greater than 5 micrometres and less than 40 micrometres.

To modify the effect of the wall to constrain or contain the adherent sample, at least a portion of the wall may have a rectangular cross section, or may have a flared or rounded cross section. For example, the cross-section of at least a portion of the wall may be stepped, or its thickness may vary non-monotonically. In particular, the cross-section of the wall may be flared such that a cross-section of the wall further from the sample surface is greater than the cross-section of the wall nearest to the sample surface. For example a portion of the wall may overhang the sample surface. This may improve the containment of the adherent sample. Alternatively, the cross-section of the wall may be flared such that the cross-section of the wall further from the sample surface is smaller than the cross-section of the wall nearest to the sample surface. This may allow the adherent cells to reach the sample surface of the base portion more easily. The wall preferably has the same cross section throughout its length.

Flared walls where the top is thicker than the base of the walls, or walls which overhang the sample surface, may advantageously provide a mechanical barrier to cell movement away from the sample surface. Alternatively, flared walls where the top of the wall is less thick than the base of the walls may advantageously allow any cells which may adhere to the wall to migrate down to the sample surface. The wall shape may thus be optimised for different cell or sample types, and different wall and sample-surface coatings.

Advantageously, the carrier may have a lateral dimension, preferably a minimum lateral dimension, of greater than 5 micrometres and less than 300 micrometres, or preferably greater than 10 micrometres and less than 200 micrometres.

The wall of the carrier may be fabricated by any suitable method, but advantageously, for convenience and efficiency, the wall may comprise a material which is also present in the base portion, and the wall is preferably fabricated using a process which is also used to fabricate the base portion. For example, the wall and the base portion may conveniently be fabricated using a lithographic process, in which case a magnetic material can also be incorporated into the carrier.

The wall may be fabricated using nanoimprint lithography or micro-imprint lithography.

In one embodiment, particularly if the carrier is made using photolithography, the wall may comprise a structural photopolymer, preferably a photoresist, SU-8 or AZ-10nXT. Alternatively, or in addition, the wall may comprise a polymer, such as a thermoplastic polymer, preferably PDMS.

In a further aspect, the invention may advantageously provide a carrier system for an assay, comprising a plurality of carriers. In the carrier system, the carriers are preferably provided secured to a substrate by a release layer as described above. The substrate surface to which the carriers are secured is preferably flat.

In yet a further aspect, the invention may advantageously provide a method of performing an assay using the carrier or the carrier system as described above. The method may advantageously comprise the step of providing a plurality of carriers positioned on a substrate, introducing a biocompatible aqueous solution carrying the adherent samples to the carriers and the substrate so that the adherent samples adhere to the sample surfaces of the carriers, and releasing the carriers from the substrate. The carriers may be held in position on the substrate by a release layer between the carriers and the substrate, the release layer being activated by the biocompatible aqueous solution to release the carriers. In addition, or in the alternative, the carriers may be magnetic or comprise a magnetic material and may then be held in position on the substrate by an applied magnetic field and released from the substrate by changing or removing the applied magnetic field.

In yet a further aspect of the invention, the carrier of the invention is preferably magnetically steerable, for example through the stages of an assay. Therefore, after release from the substrate, a solution in an assay may contain a large number of carriers, carrying adherent samples. While in solution, the carriers may move near to each other and it is important to avoid the adherent samples moving from one carrier to another. The geometrical effect of the walls, preferably surrounding the adherent samples on each carrier, may advantageously assist in keeping the sample surfaces of different carriers spaced from each other when the carriers are in solution, and thus may reduce the risk of adherent samples moving from one carrier to another.

In some cases, individual carriers may be marked using a readable code, such as a barcode, or otherwise be marked so that individual carriers, or groups of carriers, can be identified in an assay. If adherent samples are placed on an individual carrier at the start of an assay, then it is then essential that those samples do not move from one carrier to another. The walls surrounding the samples on carriers in solution, which may move near to or past other carriers, may advantageously prevent movement of samples between carriers.

The carrier may comprise a readable code applied to the sample surface of the carrier. Alternatively, the readable code may comprise a hole or a number of holes, or a region or regions of different materials, defined through or within the base portion of the carrier. Preferably, the wall comprises a readable code. Advantageously, the readable code being comprised in the wall prevents the code from reducing the area of the sample surface available for cell attachment. The code may comprise a number of dots, dashes, or dots and dashes. The code may comprise a number of lines. The code may penetrate partially into the wall. The code may penetrate through the wall to the carrier base. The code may be formed by lithography.

The readable code may take a variety of forms as markings in the carrier, which encode information identifying the carrier to which that code is applied. When imaged, the pattern in the base or walls of a given carrier can be analysed for its coding information, and thus used to identify that specific carrier. The code is preferably readable by image analysis of a microscopy image.

Also, in an assay, magnetically-steerable carriers may commonly be steered onto a substrate, for example for imaging or analysis. When the carriers are placed onto a substrate in this way, the walls may advantageously impede adherent samples from moving from the carriers to the substrate, or from one carrier to another. In addition, as noted above, carriers which are moving in a solution during an assay may move near to or even into contact with other carriers, and the walls may advantageously prevent movement of samples between such carriers. These effects can only be achieved by placing the walls on the carriers themselves. In the prior art, walls have been used to locate or constrain adherent samples on a substrate, but the prior-art walls are secured to the substrate and can therefore have no effect after carriers, such as magnetically-steerable carriers, have been moved or steered to a new substrate in an assay.

In addition, as the carriers are driven through the assay medium by an external magnetic field, the assay samples carried by the carriers are subjected to shear forces. These forces may be detrimental to the assay samples, and the presence of a wall adjacent to the assay samples on the sample surface of the carrier may advantageously reduce the shear forces experienced by the assay samples.

The invention is defined in the claims. However, below there is provided a non-exhaustive list of non-limiting examples. Any one or more of the features of these examples may be combined with any one or more features of another example, embodiment, or aspect described herein.

### EXAMPLES (EX)

EX1. A carrier for an adherent assay sample, which comprises a sample surface for receiving the adherent sample and a peripheral region surrounding the sample surface and positioned between the sample surface and an edge of the carrier, in which the carrier comprises a surface treatment such that the adherent sample adheres less strongly to the peripheral region than to the sample surface.

EX2. A carrier according to example EX1, in which during use the carrier is removably positionable on a substrate for receiving the adherent assay sample, in which when the carrier is positioned on the substrate the peripheral region is between the sample surface and the substrate, preferably encircling the sample surface.

EX3. A carrier according to any preceding example, in which when the carrier is positioned on the substrate the sample surface and the peripheral region differ in height above the substrate by less than 10% of a maximum thickness of the carrier, and preferably by less than 1% or 0.1% of the maximum thickness of the carrier.

EX4. A carrier according to any preceding example, in which a thickness of the carrier in the region of the sample surface and a thickness of the carrier in the peripheral region differ in by less than 10%, and preferably by less than 1% or 0.1%.

EX5. A carrier according to any preceding example, in which the peripheral region is substantially flat, and is preferably parallel to the substrate when the carrier is positioned on the substrate.

EX6. A carrier according to any preceding example, in which the sample surface is substantially flat, and is preferably parallel to the substrate when the carrier is positioned on the substrate.

EX7. A carrier according to any preceding example, in which the carrier comprises a base portion, and an upper surface of the base portion forms the sample surface and the peripheral region.

EX8. A carrier according to any preceding example, in which the peripheral region comprises a sample-adhesion-reducing coating.

EX9. A carrier according to example EX8, in which the coating on the peripheral region comprises a non-ionic surfactant.

EX10. A carrier according to example EX8 or EX9, in which the coating on the peripheral region comprises one or more selected from Pluronic F-127, Poly-HEMA, a PEG polymer, and a Poloxamer.

EX11. A carrier according to any preceding example, in which the sample surface comprises a sample-adhesion-promoting coating.

EX12. A carrier according to example EX11, in which the sample surface comprises a bio-functionalised coating adapted to receive the adherent sample.

EX13. A carrier according to example EX11 or EX12, in which the sample surface comprises a charged polymer.

EX14. A carrier according to any preceding example, in which at least a portion of the peripheral region has a lateral width, or a maximum lateral thickness, of greater than 1 micrometre and less than 50 micrometres, or greater than 5 micrometres and less than 40 micrometres.

EX15. A carrier according to any preceding example, in which the carrier has a lateral dimension, preferably a minimum lateral dimension, of greater than 5 micrometres and less than 300 micrometres, or preferably greater than 10 micrometres and less than 200 micrometres.

EX16. A carrier according to any preceding example, in which the carrier comprises a magnetic material.

EX17. A carrier system for an assay, comprising a plurality of carriers as defined in any of examples EX1 to EX16.

EX18. A method of performing an assay using the carrier as defined in any of examples EX1 to EX16 or using the carrier system as defined in EX17, the method comprising the step of providing a plurality of carriers positioned on a substrate, introducing a biocompatible aqueous solution carrying the adherent samples to the carriers and the substrate so that the adherent samples adhere to the sample surfaces of the carriers but not to the peripheral region, and releasing the carriers from the substrate.

EX19. A method of performing an assay according to example EX18, in which the carriers are held in position on the substrate by a release layer between the carriers and the substrate, the release layer being activated by the biocompatible aqueous solution to release the carriers.

EX20. A method of performing an assay according to example EX18 or EX19, in which the carriers are magnetic or comprise a magnetic material and are held in position on the substrate by an applied magnetic field and released from the substrate by changing or removing the applied magnetic field.

EX21. A carrier for an adherent assay sample, which comprises a sample surface for, in use, receiving the adherent sample and a wall between the sample surface and an edge of the carrier.

EX22. A carrier according to example EX21, in which the adherent sample adheres, in use, more strongly to the sample surface than to the wall.

EX23. A carrier according to example EX21 or EX22, in which during use the carrier is removably positionable on a substrate for receiving the adherent assay sample, in which when the carrier is positioned on the substrate the wall is between the sample surface and the substrate.

EX24. A carrier according to any one of examples EX21 to EX23, in which the wall encircles the sample surface.

EX25. A carrier according to any one of examples EX21 to EX24, in which the sample surface is substantially flat, and is preferably parallel to the substrate when the carrier is positioned on the substrate.

EX26. A carrier according to any one of examples EX21 to EX25, in which the carrier comprises a base portion, an upper surface of the base portion forms the sample surface, and the wall extends above the upper surface of the base portion, adjacent to or spaced from the sample surface.

EX27. A carrier according to any one of examples EX21 to EX26, in which at least a portion of the sample surface comprises a coating such that the adherent sample adheres more strongly to the sample surface than to the wall.

EX28. A carrier according to any one of examples EX21 to EX27, in which at least a portion of a surface of the wall comprises a coating such that the adherent sample adheres less strongly to the wall than to the sample surface.

EX29. A carrier according to any one of examples EX21 to EX28, in which at least a portion of the wall has a height above the sample surface of greater than 1 micrometre and less than 25 micrometres.

EX30. A carrier according to any one of examples EX21 to EX29, in which at least a portion of the wall has a lateral thickness, or a maximum lateral thickness, of greater than 1 micrometre and less than 40 micrometres.

EX31. A carrier according to any one of examples EX21 to EX30, in which at least a portion of the wall has a rectangular cross section, or has a flared or rounded cross section.

EX32. A carrier according to any one of examples EX21 to EX31, in which the carrier has a lateral dimension, preferably a minimum lateral dimension, of greater than 5 micrometres and less than 300 micrometres, or preferably greater than 10 micrometres and less than 200 micrometres.

EX33. A carrier according to any one of examples EX21 to EX32, in which the carrier comprises a magnetic material.

EX34. A carrier according to any one of examples EX21 to EX33, in which the carrier comprises a base portion, an upper surface of which forms the sample surface, and in which the wall comprises a material which is also present in the base portion, and is preferably fabricated using a process which is also used to fabricate the base portion.

EX35. A carrier according to any one of examples EX21 to EX34, in which the carrier comprises a base portion, an upper surface of which forms the sample surface, and in which the wall and the base portion are fabricated using a lithographic process.

EX36. A carrier according to any one of examples EX21 to EX35, in which the wall comprises a structural photopolymer, preferably a photoresist, SU-8 or AZ-10nXT.

EX37. A carrier according to any one of examples EX21 to EX36, in which the wall comprises a polymer, such as a thermoplastic polymer, preferably PDMS.

EX38. A carrier according to any one of examples EX21 to EX37, in which the sample surface comprises a gold cap layer on to which a polymer is covalently bonded by a thiol group.

EX39. A carrier system for an assay, comprising a plurality of carriers as defined in any of examples EX21 to EX38.

EX40. A method of performing an assay using the carrier as defined in any of examples EX21 to EX38 or using the carrier system as defined in example EX39, the method comprising the step of providing a plurality of carriers positioned on a substrate, introducing a biocompatible aqueous solution carrying the adherent samples to the carriers and the substrate so that the adherent samples adhere to the sample surfaces of the carriers, and releasing the carriers from the substrate.

EX41. A method of performing an assay according to example EX40, in which the carriers are held in position on the substrate by a release layer between the carriers and the substrate, the release layer being activated by the biocompatible aqueous solution to release the carriers.

EX42. A method of performing an assay according to example EX40 or EX41, in which the carriers are magnetic or comprise a magnetic material and are held in position on the substrate by an applied magnetic field and released from the substrate by changing or removing the applied magnetic field.

### SPECIFIC DESCRIPTION

Specific embodiments of the invention will now be described by way of example, with reference to the accompanying drawings in which:

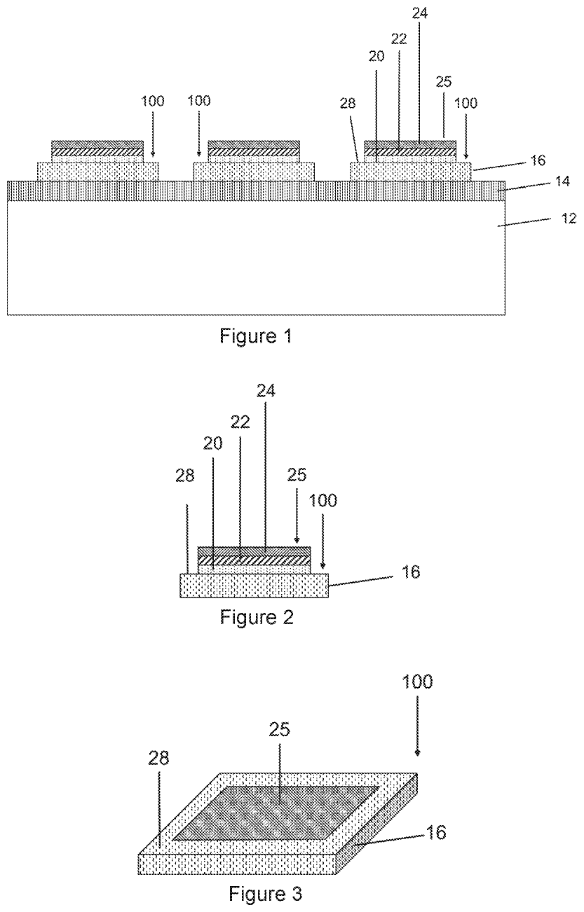

FIG. 1 is a schematic sectional view of three carriers secured to a substrate, according to a first embodiment of the invention;

FIG. 2 is a schematic sectional view of a carrier as in FIG. 1, after release from the substrate;

FIG. 3 is a schematic three-quarter view of a carrier as in FIGS. 1 and 2;

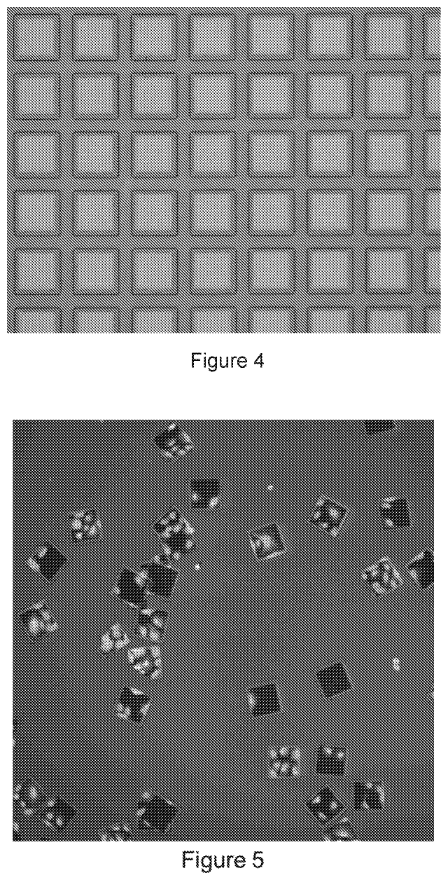

FIG. 4 is a photomicrograph of an array of carriers embodying the invention, secured to a substrate;

FIG. 5 is a photomicrograph of a number of carriers embodying the invention carrying adherent cells and suspended in an assay solution;

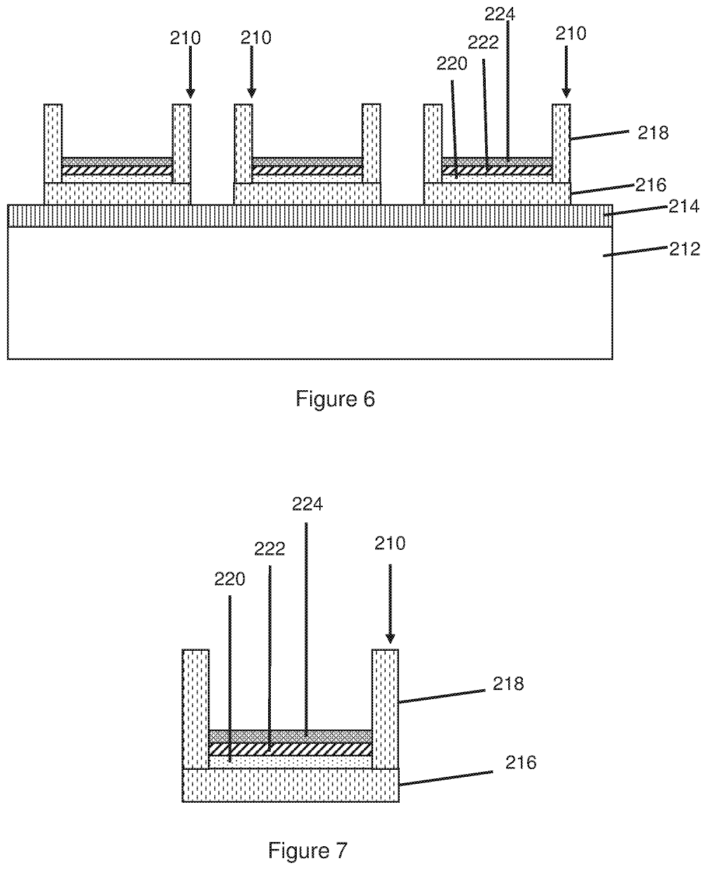

FIG. 6 is a schematic sectional view, with vertical dimensions enlarged for clarity, of three carriers according to a second embodiment of the invention, attached by a release layer to a substrate;

FIG. 7 is a schematic sectional view of a carrier of FIG. 6 after release from the substrate;

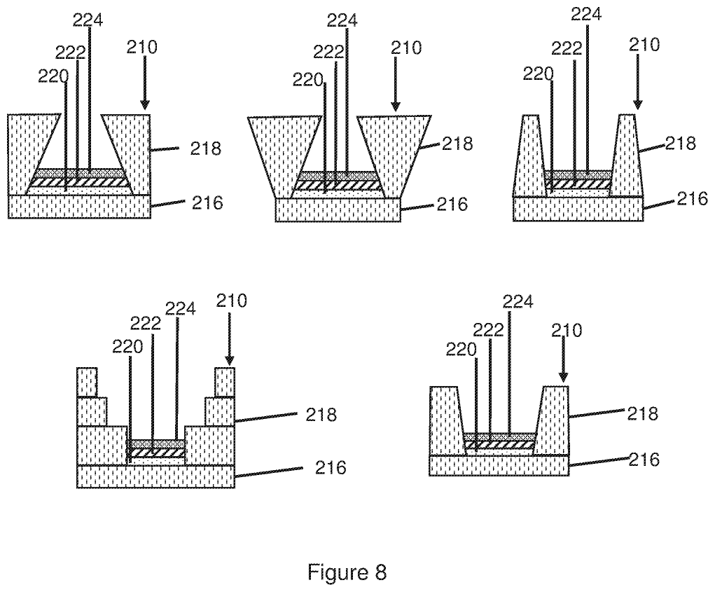

FIG. 8 shows schematic sectional views of five alternative embodiments of the invention, similar to the carrier of FIG. 6 after release from the substrate, with carrier walls of different cross-sectional shapes;

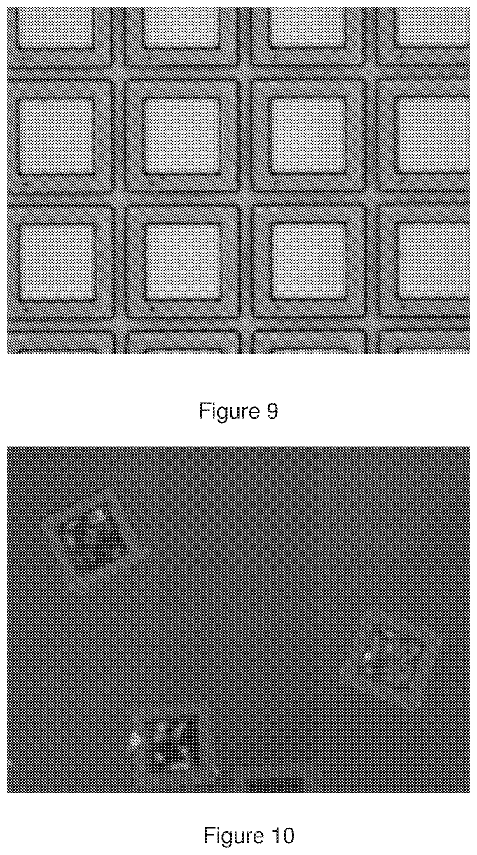

FIG. 9 is a photomicrograph plan view of a substrate supporting an array of the carriers of FIGS. 6 and 7; and

FIG. 10 is a photomicrograph of carriers as in the second embodiment, carrying adherent cells and suspended in an assay solution.

A carrier according to a first embodiment of the invention is shown in FIGS. 1, 2 and 3. As shown in FIG. 1, carriers 100 are initially provided for an assay, secured to a substrate 12 in the form of a silicon chip by a release layer 14, which may comprise a dextran. The substrate is sized to carry a plurality of perhaps 1000 to 500,000 carriers, depending on the assay application. Each carrier is suitable for receiving an adherent cell or a plurality of adherent cells.

The (or each) carrier 100 is fabricated as follows. The release layer 14 is spin-coated over the silicon substrate 12. A square layer 16 of a structural photopolymer is then deposited onto the release layer. The shape of the photopolymer is defined by photolithography, and is about 1.5 micrometres thick with a lateral dimension along each side of the square of 120 micrometres The photopolymer, which may be SU-8, is therefore in the shape of a flat, or high-aspect-ratio, cuboid with relatively large, square upper and lower surfaces. The square layer of photopolymer forms the base, or body, of the carrier.

On a square central portion of an upper surface of the layer 16 of photopolymer, a multi-layered magnetic heterostructure 20 is deposited using lithography techniques as described in WO2020/099846. The magnetic heterostructure is only deposited on a central portion of the layer of photopolymer to define the shape of the sample surface.

A functionalisable gold cap 22 is then deposited onto the multi-layered magnetic heterostructure 20, followed by a layer of a polymer 24 to which the target adherent samples can readily adhere. This polymer layer 24 defines a sample surface 25 of the carrier for receiving the assay sample, when an assay is performed. The sample surface is centrally positioned on the carrier base, and is square in shape with sides of about 100 micrometres.

In this embodiment, the peripheral region 28 surrounding the sample surface is 10micrometres wide.

The polymer of the polymer layer 24 comprises a thiol group and is applied by immersing the carrier and substrate in a solution comprising the polymer. The polymer covalently bonds to the gold cap 22 of the carrier by the thiol group. The solution comprises a non-aqueous solvent such as ethanol. The release layer is non-soluble in such a non-aqueous solvent and so the carrier remains secured to the substrate while immersed in the solution comprising the polymer.

The cumulative thickness of the magnetic heterostructure, the gold cap and the polymer layer is less than 1 micrometre and is typically a few hundred nanometres, depending in particular on the thickness of the attachment polymer layer. Therefore, the upper surface of the completed carrier is substantially flat, or planar.

Each carrier may comprise a readable code such as a barcode. Predetermined codes are assigned to each carrier to identify the carriers. For example, the predetermined code can refer to a particular adherent cell received on the carrier or a particular adherent cell and particular reagent that the adherent cell has been exposed to as part of an assay. In other words, the codes allow for a multichannel assay to be performed by a plurality of the carriers. The code may be defined in the peripheral region of the carrier. In an example the code comprises a number of dots or dashes which are defined in the peripheral region by photolithography, for example by incorporating holes or indents in the SU-8 base layer in a single exposure.

Carriers embodying the present invention may also be realised by forming the magnetic heterostructure spanning the entire carrier and then defining the area and shape of the sample surface 25 as a portion of the upper surface of the magnetic heterostructure. This may be done by depositing the gold cap or the polymer coating in a selected area of the carrier surface, surrounded by a peripheral region 28 which extends around the edges of the carrier surface. The sample surface thus occupies a relatively central position on the carrier surface, with the peripheral region 28 extending all of the way around the sample surface, spanning the area between the sample surface and the carrier edge.

In further preferred embodiments the sample surface 25 is about 100 micrometres square, and the width of the peripheral region is roughly 20 micrometres between the edge of the carrier and the edge of the sample surface.

The sample surface 25 and the peripheral region 28 may be coated differently, or may be of different materials or receive different materials treatments, so that an assay sample adheres more strongly to the sample surface 25 than to the peripheral region 28.

Thus, for example, after forming the sample surface 25 as described above, the perimeter region 28 of the upper surface of the photopolymer layer 16 is coated in an adhesion-reducing coating material, to reduce or prevent adhesion of assay sample (for example sample cells) to the peripheral region 28 of the carrier 10. The coating of the peripheral region can be formed by depositing a non-ionic surfactant such as Pluronic F-127 in a strip surrounding the central sample surface. The sample surface may, for example, be coated with a gold cap on which the low-adhesion surface treatment would not form.

In different embodiments, instead of lithographically depositing the gold cap 22 on only the central sample surface 25, the gold cap may be formed over the entire upper surface of the carrier 100, and the respective coating or coatings which define the sample surface may be applied only to defined areas on the gold cap. For example, the gold cap may be functionalised only in the sample surface area in the centre of the carrier, while an adhesion-reducing coating may be applied to the exposed part of the gold cap around the perimeter of the carrier, to surround the sample surface.

In other embodiments, coatings other than the polymer coating 24 described above may be used to coat the sample surface. For example, the adhesion-promoting sample surface coating can comprise a plurality of ligands comprising antibodies that specifically bind to cell receptors such as integrins or an extracellular matrix protein such as collagen, or Matrigel, a protein mixture secreted by Engelbreth-Holm-Swarm (EHS) mouse sarcoma cells. Alternatively to applying a coating to the sample surface, the method can comprise modifying the physical surface of the sample surface to be particularly suitable for receiving adherent cells, for example by modifying the gold cap.

In other embodiments, a variety of adhesion-reducing coatings may be used to reduce adhesion of sample to the peripheral regions 18, 28. For example the peripheral region may be coated with Poly-HEMA, or another Poloxamer.

FIG. 4 is a plan view photomicrograph of an array of carriers secured to a substrate. Any number of carriers can be secured to the substrate as required for any assay application.

The final step of the method of manufacturing the carrier system is sterilization, not shown in the figures. In order to sterilize the carrier system, an array of carriers secured to a substrate is put into sterilization packaging, sealed, and then placed in an oven at 120degrees Celsius for 4 hours. This sterilizes it and creates a ready-to-use sample that can be opened in a sterile cell culture environment.

FIG. 5 is a photomicrograph of a plurality of carriers embodying the invention in use in an assay of adherent cells, suspended in the assay solution. For the photomicrograph, the magnetic carriers have been aligned facing the camera by suitable alignment of an external magnetic field, and the cells have been stained to improve their visibility. It can be seen that the cells adhere to the sample surfaces of each carrier, and do not adhere to the peripheral region surrounding the sample surface of each carrier.

To place the cells on the sample surfaces of the carriers in FIG. 5, the following steps have been taken. A carrier system such as the array of carriers on a silicon substrate has been placed in a container and immersed in a biocompatible aqueous solution comprising the adherent cells. When the biocompatible aqueous solution comes into contact with the release layer 14, the release layer 14 dissolves and releases the carriers. If additional time is needed for the cells to adhere to the carriers while the carriers are on the substrate, an external magnetic field is applied to retain the carriers in contact with the substrate even after the release layer has released the carriers.

Once the cells have adhered to the carriers, the carriers are released from substrate, and are free to move through the aqueous solution surrounding the carrier system. External magnetic fields can be used to apply forces to carriers in order to manipulate and move them as desired to perform the assay. As shown in FIG. 5, the magnetic field can be used to align the carriers for photographic imaging.

The structure of a carrier 210 according to a second embodiment of the invention is illustrated in FIGS. 6 and 7. FIG. 6 shows three carriers secured to a substrate 212, in the form of a silicon chip, by a release layer 214. The substrate is sized to receive a plurality of perhaps between 1000 and 500,000 carriers, depending on the assay application. Each carrier is suitable for receiving an adherent cell or a plurality of adherent cells.

The (or each) carrier 210 is fabricated as follows. The release layer 214, which may comprise a dextran, is spin-coated over the silicon substrate 212. A square layer 216 of a structural photopolymer is then deposited onto the release layer. The shape of the photopolymer is defined by photolithography, and is about 1.5 micrometres thick with a lateral dimension along each side of the square of 160 micrometres. The photopolymer, which may comprise SU-8, is therefore in the shape of a flat, or high-aspect-ratio, cuboid with relatively large, square upper and lower surfaces. An upstanding wall 218 of the photopolymer is then formed along the edges of the square layer 216, again by photolithography. The wall is 7 micrometres high and 20 micrometres thick.

A surface treatment is then applied to at least a portion of the wall which reduces adhesion of adherent samples to the walls. The coating may be done for example by immersing in an ethanol solution containing Pluronic F127 which binds to the hydrophobic photopolymer comprising the walls, or by treating the wall surface with an oxygen plasma and immersing in a silane-PEG ethanol solution where the silane-PEG covalently binds to the oxygen plasma activated surface. (By contrast, in a preferred embodiment the sample surface may comprise a gold layer, which may be different to the photopolymer of the wall in its hydrophobicity, and does not form surface-OH groups on plasma treatment. Therefore the low adhesion surface treatment may not be applied to the sample surface.)

In some embodiments it may be desirable to apply the low-adhesion surface treatment to only a specific portion of the wall. This may be achieved by masking. For example a sacrificial mask of photoresist may be patterned before the low-adhesion surface treatment is applied, to block certain areas of the walls from being coated.

Optionally, in some embodiments it may be desirable additionally to apply a surface treatment to the substrate between the carriers, to reduce any adhesion of samples to the substrate. This may only be required for certain uses of the carrier system, where adhesion of samples to the substrate may be a problem. The exposed release layer on the substrate dissolves in use when the carrier system is exposed to a biocompatible aqueous solution carrying the adherent samples and so, in order to apply a surface treatment between the carriers, the exposed release layer may be removed from the substrate and a surface treatment such as Pluronic 127 or silane-PEG be applied to the substrate between the carriers.

On an upper surface of the layer 216 of photopolymer, within the walls 218, a multi-layered magnetic heterostructure 220 is then deposited (as described in WO2020/099846).

A functionalisable gold cap 222 is then deposited onto the multi-layered magnetic heterostructure 220, followed by a layer of a polymer 224 to which the target adherent samples can readily adhere. This polymer layer 224 defines a sample surface, which is about 100 micrometres square, of the carrier for receiving the assay sample when an assay is performed.

The polymer of the polymer layer 224 comprises a thiol group and is applied by immersing the carrier and substrate in a solution comprising the polymer. The polymer covalently bonds to the gold cap 222 of the carrier by the thiol group. The solution comprises a non-aqueous solvent such as ethanol. The release layer is non-soluble in such a non-aqueous solvent and so the carrier will remain secured to the substrate while immersed in the solution comprising the polymer.

The cumulative thickness of the magnetic heterostructure, the gold cap and the polymer layer is less than 1 micrometre and is typically a few hundred nanometres, depending in particular on the thickness of the attachment polymer layer, so that the walls extend about 6 to 7 micrometres above the sample surface.

Each carrier may comprise a readable code such as a barcode. Predetermined readable codes are assigned to each carrier to identify the carriers. For example, a predetermined barcode can then identify a carrier carrying a particular adherent cell, as well as any particular reagent that the adherent cell has been exposed to as part of an assay. In other words, the codes allow for a multichannel assay to be performed by a plurality of the carriers. The code may be defined in the wall of the carrier. In an embodiment, for example, a barcode of dots and dashes, or lines, can be included in the lithographic pattern for the walls. This may be as simple as the number of dots, dashes, or lines, or more complex as in an actual barcode. When imaged, the pattern in the walls of a given carrier can be analysed for its barcoding information.

As noted above, in other embodiments, different shapes or cross-sections of the walls may be appropriate for different cell or sample types, and different wall and sample-surface coatings. Some examples are illustrated in FIG. 8.

The first two examples in FIG. 8 (reading from left to right) show flared walls where the top is thicker than the base of the walls, so that the walls overhang the sample surface. At the same time the outer surface of the walls may be vertical, perpendicular to the base of the carrier, or may be outwardly flared. Such walls may advantageously provide a mechanical barrier to cell movement away from the sample surface and may restrict movement of cells towards the substrate between carriers.

Alternatively, as shown at the top right and bottom right of FIG. 8, flared walls where the top of the wall is less thick than the base of the wall may be used. Such wall shapes may advantageously encourage any cells which may adhere to the wall to migrate down to the sample surface.

In variations of these wall shapes, the wall cross-section may be stepped, as shown at the lower left of FIG. 8. Stepped wall shapes may be easier to fabricate than continuously flared walls, while retaining the functional advantages of flared walls.

Walls may combine features of these wall shapes. For example, a wall may have an inwardly-flared inner surface (overhanging the sample surface) and an inwardly-flared outer surface (sloping away from the surrounding substrate), or vice versa.

The wall shape may thus be optimised for different cell or sample types, and different wall and sample-surface surface treatments.

FIG. 9 is a plan view of an array of carriers, according to the second embodiment, secured to a substrate. Any number of carriers can be secured to the substrate depending on the assay application.

In other embodiments, surface treatments other than the polymer coating described above for the sample surface may be used. For example, the coating can comprise a plurality of ligands comprising antibodies that specifically bind to cell receptors such as integrins or an extracellular matrix protein such as collagen, or Matrigel, a protein mixture secreted by Engelbreth-Holm-Swarm (EHS) mouse sarcoma cells. Alternatively to applying a coating, the method can comprise modifying the physical surface of the sample surfaces of the carriers to be particularly suitable for receiving adherent cells, for example by modifying the gold cap.

The final step of the method of manufacturing the carrier system is sterilization, not shown in the figures. In order to sterilize the carrier system, it is put into sterilization packaging, sealed, and then placed in an oven at 120 degrees Celsius for 4 hours. This sterilizes it and creates a ready-to-use sample that can be opened in a sterile cell culture environment.

FIG. 10 is a photomicrograph of a plurality of the carriers of the second embodiment in use in an assay of adherent cells, suspended in the assay solution. For the photomicrograph, the magnetic carriers have been aligned facing the camera by suitable alignment of an external magnetic field, and the cells have been stained to improve their visibility. It can be seen that the cells adhere to the sample surfaces of each carrier, and do not adhere to the wall surrounding the sample surface of each carrier.

To place the cells on the sample surfaces of the carriers in FIG. 10, the following steps have been taken. A carrier system such as the array of carriers on a silicon substrate shown in FIG. 9 has been placed in a container and immersed in a biocompatible aqueous solution comprising the adherent cells. When the biocompatible aqueous solution comes into contact with the release layer 214, the release layer 214 dissolves and releases the carriers. If additional time is needed to allow the cells to adhere to the carriers while the carriers are on the substrate, an external magnetic field is applied to retain the carriers in contact with the substrate even after the release layer has released the carriers.

Once the cells have adhered to the carriers, the carriers are released from the substrate, and are free to move through the aqueous solution surrounding the carrier system. External magnetic fields can be used to apply forces to the carriers in order to manipulate and move them as desired to perform the assay. As shown in FIG. 10, the magnetic field can be used to align carriers for photographic imaging.

1. A carrier for an adherent assay sample, which comprises a sample surface for receiving the adherent sample and a peripheral region surrounding the sample surface and positioned between the sample surface and an edge of the carrier, in which the peripheral region comprises a sample-adhesion-reducing coating, such that, in use, the adherent sample adheres less strongly to the peripheral region than to the sample surface.

2. A carrier according to claim 1, in which during use the carrier is removably positionable on a substrate for receiving the adherent assay sample, in which when the carrier is positioned on the substrate the peripheral region is between the sample surface and the substrate, preferably encircling the sample surface.

3. A carrier according to claim 2, in which when the carrier is positioned on the substrate the sample surface and the peripheral region differ in height above the substrate by less than 10% of a maximum thickness of the carrier, and preferably by less than 1% or 0.1% of the maximum thickness of the carrier.

4. A carrier according to any preceding claim, in which a thickness of the carrier in the region of the sample surface and a thickness of the carrier in the peripheral region differ by less than 10%, and preferably by less than 1% or 0.1%.

5. A carrier according to any preceding claim, in which the peripheral region is substantially flat, and is preferably parallel to the substrate when the carrier is positioned on the substrate.

6. A carrier according to any preceding claim, in which the sample surface is substantially flat, and is preferably parallel to the substrate when the carrier is positioned on the substrate.

7. A carrier according to any preceding claim, in which the carrier comprises a base portion, and an upper surface of the base portion forms the sample surface and the peripheral region.

8. A carrier according to any preceding claim, comprising a wall between the sample surface and an edge of the carrier.

9. A carrier according to claim 8, in which the peripheral region comprises the wall.

10. A carrier according to claim 8 or 9, in which at least a portion of a surface of the wall comprises the sample-adhesion-reducing coating such that, in use, the adherent sample adheres less strongly to the wall than to the sample surface.

11. A carrier according to any one of claims 8 to 10, in which the wall encircles the sample surface.

12. A carrier according to any one of claims 8 to 11, in which when the carrier is positioned on the substrate the wall is between the sample surface and the substrate.

13. A carrier according to any one of claims 8 to 12, in which the carrier comprises a base portion, an upper surface of which forms the sample surface, and wherein the wall extends above the upper surface of the base portion, adjacent to or spaced from the sample surface.

14. A carrier according to any one of claims 8 to 13, in which at least a portion of the wall has a height above the sample surface of greater than 1 micrometre and less than 25 micrometres.

15. A carrier according to any one of claims 8 to 14, in which at least a portion of the wall has a lateral thickness, or a maximum lateral thickness, of greater than 1 micrometre and less than 40 micrometres.

16. A carrier according to any one of claims 8 to 15, in which at least a portion of the wall has a rectangular cross section, or has a flared or rounded cross section.

17. A carrier according to any one of claims 8 to 16, in which the carrier comprises a base portion, an upper surface of which forms the sample surface, and in which the wall comprises a material which is also present in the base portion, and is preferably fabricated using a process which is also used to fabricate the base portion.

18. A carrier according to any one of claims 8 to 17, in which the carrier comprises a base portion, an upper surface of which forms the sample surface, and in which the wall and the base portion are fabricated using a lithographic process.

19. A carrier according to any one of claims 8 to 18, in which the wall comprises a structural photopolymer, preferably a photoresist, SU-8 or AZ-10nXT.

20. A carrier according to any one of claims 8 to 19, in which the wall comprises a polymer, such as a thermoplastic polymer, preferably PDMS.

21. A carrier according to any preceding claim, in which the coating of the peripheral region comprises a non-ionic surfactant.

22. A carrier according to any preceding claim, in which the coating of the peripheral region comprises one or more selected from Pluronic F-127, Poly-HEMA, a PEG polymer, and a Poloxamer.

23. A carrier according to any preceding claim, in which the sample surface comprises a sample-adhesion-promoting coating.

24. A carrier according to claim 23, in which the sample surface comprises a bio-functionalised coating adapted to receive the adherent sample.

25. A carrier according to claim 23 or 24, in which the sample surface comprises a charged polymer.

26. A carrier according to any preceding claim, in which at least a portion of the peripheral region has a lateral width, or a maximum lateral thickness, of greater than 1 micrometre and less than 50 micrometres, or greater than 5 micrometres and less than 40 micrometres.

27. A carrier according to any preceding claim, in which the carrier has a lateral dimension, preferably a minimum lateral dimension, of greater than 5 micrometres and less than 300 micrometres, or preferably greater than 10 micrometres and less than 200 micrometres.

28. A carrier according to any preceding claim, in which the carrier comprises a magnetic material.

29. A carrier system for an assay, comprising a plurality of carriers as defined in any of claims 1 to 28.

30. A method of performing an assay using the carrier as defined in any of claims 1 to 28 or using the carrier system as defined in claim 29, the method comprising the step of providing a plurality of carriers positioned on a substrate, introducing a biocompatible aqueous solution carrying the adherent samples to the carriers and the substrate so that the adherent samples adhere to the sample surfaces of the carriers but not to the peripheral regions, and releasing the carriers from the substrate.

31. A method of performing an assay according to claim 30, in which the carriers are held in position on the substrate by a release layer between the carriers and the substrate, the release layer being activated by the biocompatible aqueous solution to release the carriers.

32. A method of performing an assay according to claim 30 or 31, in which the carriers are magnetic or comprise a magnetic material and are held in position on the substrate by an applied magnetic field and released from the substrate by changing or removing the applied magnetic field.
